(19) United States
(12) Patent Application Publication
(10) Pub. No.: US 2022/0214237 A1
(43) Pub. Date: Jul. 7, 2022

(54) DEVICE FOR MEASURING A TORQUE AND STRAIN WAVE GEARING COMPRISING SUCH A DEVICE

(71) Applicant: Schaeffler Technologies AG & Co. KG, Herzogenaurach (DE)

(72) Inventors: Jürgen Gierl, Erlangen (DE); Philipp Hörning, Bamberg (DE); Romina Baechstaedt, Niederndorf-Herzogenaurach (DE); Jens Heim, Bergrheinfeld (DE); Ricardo Henrique Brugnara, Heßdorf (DE); Jochen Damerau, Tokyo (JP)

(21) Appl. No.: 17/607,369

(22) PCT Filed: Apr. 29, 2020
(86) PCT No.: PCT/DE2020/100350

§ 371 Date: Oct. 28, 2021

(30) Foreign Application Priority Data
May 9, 2019 (DE) 10 2019 112 146.9

(51) Int. Cl. G01L 3/10; F16H 49/00; B25J 9/10; B25J 13/08

(52) U.S. Cl. CPC G01L 3/108; F16H 49/001; F16H 2049/003; B25J 13/085; B25J 9/1025

(57) ABSTRACT

A device for measuring a torque of a strain wave gearing includes a component (01, 02), on which the torque is applied, an electrically insulating insulation layer (06) arranged on the component (01, 02) and a deformation-sensitive measurement layer (04) arranged on the insulation layer (06). A strain wave gearing for a robot arm has such a device for measuring a torque.

This application is the U.S. National Phase of PCT Appln. No. PCT/DE2020/100350 filed Apr. 29, 2020, which claims priority to DE 10 2019 112 146.9 filed May 9, 2019, the entire disclosures of which are incorporated by reference herein.

The present disclosure relates to a device for measuring a torque occurring in a strain wave gearing of a robot. In particular, the device is used in robot joints. The present disclosure further relates to a strain wave gearing.

### BACKGROUND

A measuring device for determining a torque acting on an axis is known from DE 10 2010 029 186 A1, wherein the measuring device comprises a first and a second device. The devices are each designed to generate an analog electrical signal associated with the torque. Two independent torques are determined by means of downstream analog-to-digital converters and downstream digital evaluation devices. The devices are made up of strain gages applied to mechanical measuring bodies.

DE 10 2014 210 379 B4 describes a torque sensor and a method for measuring torques occurring at or in a joint of a jointed-arm robot. The sensor comprises a plurality of measuring spokes, which are designed in such a way that they deform under the action of a torque. The sensor also comprises strain gages which are arranged on the measuring spokes.

DE 10 2012 208 492 A1 describes a method for producing a strain gage arrangement on the surface of a machine element. A deformation-sensitive measurement layer with an overlying protective layer is applied to the surface. The protective layer is removed locally by laser processing and the exposed measurement layer is electrically contacted. Furthermore, it can be gathered from this publication that an insulation layer can be arranged between the surface of the machine element and the measurement layer.

DE 10 2014 219 737 A1 describes a device for measuring a torque applied to a rotatably mounted component. A carrier component is arranged on the component, on which a deformation-sensitive material is applied as a coating. The deformation-sensitive material forms a torque measurement arrangement.

A method and a device for determining an output torque of an electric motor are known from DE 103 17 304 A1. A gear with a ring gear is arranged downstream of the electric motor. A dynamic motor torque is measured by means of a torque sensor, which is supported in a fixed position on the ring gear.

DE 10 2013 204 924 A1 describes an arrangement for determining a torque acting on a shaft. In particular, the arrangement is part of a steering column of a vehicle. The arrangement comprises a first steering shaft section on the side of the steering wheel, a second steering shaft section on the side of the steering gear, and a torsion section connecting the steering shaft sections. Furthermore, the arrangement comprises a direct coating for torque measurement, which has a strain gage.

The prior art shows that for the measurement of a torque acting on a shaft, measuring arrangements are used in which strain gages are applied outside or on the shaft.

For robotic gearings, it is of great importance to accurately determine the torque transmitted by a strain wave gearing. For example, robot arms are used as prostheses for humans in medical technology, among other applications, where the robot arm must perform both precise mechanical and gross mechanical movements at different speeds and with different loads during operation. The same applies to industrial robots.

Strain wave gearings are used, among other applications, as axle drives in robots, motor vehicles, in machine tools and in drives for printing machines. Torque transmitting strain wave gearings are also known as harmonic drives or harmonic gearing. A strain wave gearing commonly includes an input shaft, an elliptical disc, a flexible spline, an outer ring, an input shaft, and a housing. The flexible spline is externally toothed and the outer ring is internally toothed, with the two components arranged coaxially to one another so that the teeth mesh with one another.

Devices for torque measurement in robot arms are known, which are mounted outside the gear housing of the robot arm. For example, deformation bodies with strain gages arranged on them are arranged in the area of the robot arms, in particular the robot joints. Using the strain gages, shear strains are recorded to determine the applied torque at the robot joint.

### SUMMARY

Based on the prior art, an object of the present disclosure is to provide an improved torque measuring device which is designed to save space and which at the same time provides a high level of accuracy and robustness.

The device according to the present disclosure is used to measure a torque of a strain wave gearing. The torque measuring device comprises a component and a plurality of layers which are arranged one above the other on the component and which are part of a direct coating of strain gages. An electrically insulating insulation layer is arranged directly on the component. A deformation-sensitive measurement layer is arranged directly on the insulation layer.

The component is part of a robotics system, in particular the strain wave gearing. The component supporting the multiple layers is a flexible spline.

One advantage of the device according to the present disclosure is that it is designed to save space, since additional deformation bodies, which are only used to measure torque, are not required. Another advantage of the device is that it enables high accuracy and precision during operation and is very robust.

The component is preferably made of metal. Alternatively, the component is made of a semiconductor material. The flexible spline has a toothing on its outer radius. For example, the component may be a cylindrical steel sleeve that is flexible within the desired limits.

In a preferred embodiment, a protective layer is applied to the deformation-sensitive measurement layer, which protects the layers located below the protective layer from environmental influences. The protective layer is preferably made of an organic material. Alternatively, the protective layer is preferably made of an inorganic material.

The measurement layer is used to measure a strain or shear of the component, wherein a torque is measured.

The measurement layer preferably consists of metal or an alloy, in particular a nickel alloy. The nickel alloy is preferably a nickel-chromium alloy (NiCr).

The measurement layer preferably has a structuring. Particularly preferably, the measurement layer has a spatial structuring that forms a striped pattern. Different embodiments may, for example, have stripes in the angular range between 35° and 55° to the component longitudinal axis. Preferably, the structuring is created by means of a laser or by etching, wherein the structuring is created only after the measurement layer has been applied to the component.

The insulation layer preferably consists of one or multiple different oxides. Alternatively, the insulation layer consists of Diamond Like Carbon (DLC). The insulation layer can alternatively consist of one or more oxides and DLC. The insulation layer particularly preferably consists of Al₂O₃ (aluminum oxide) and/or SiO₂ (wollastonite).

For example, the insulating layer may be produced by a physical vapor deposition process (PVD) or a chemical assisted physical vapor deposition process (PACVD). In one embodiment, the insulation layer is produced by a combination of the PVD and PACVD processes.

Preferably, the sequence of layers applied to the component, consisting of measurement layer, insulation layer and protective layer, has a total thickness of less than 200 μm. Particularly preferably, the sequence of layers comprising the measurement layer and the insulation layer has a total thickness of less than 20 μm.

Preferably, further elements can be arranged on the component. In one embodiment, electronic components for signal preamplification and/or for signal evaluation and/or for signal transmission are arranged on the component.

In one embodiment, electrically conductive contact layers that make contact at least in sections are formed between the stripe sections.

The strain wave gearing according to the present disclosure comprises a device for measuring a torque according to the device described above with all of its embodiments. Further, the strain wave gearing comprises a drive shaft, a wave generator which may be a rolling bearing with a non-circular, e.g., elliptical, inner ring and a deformable outer ring, a ring gear, and an elastic sleeve referred to as a flexible spline. The latter component of the device exhibits external toothing and the ring gear exhibits internal toothing. The flexible spline and ring gear are arranged coaxially to each other so that the gear teeth mesh with one another. The inner ring of the wave generator is positioned on the drive shaft so that it drives the component.

The strain wave gearing preferably also has a housing in which the aforementioned gearing components are at least partially arranged.

The strain wave gearing according to the present disclosure advantageously saves installation space, since the device, and with it the coating, is arranged within the housing and no additional deformation bodies are necessary. Due to the high precision that the device provides by accurately measuring a torque, the device and the strain wave gearing are applicable and particularly advantageous in the field of robotics. In particular, the device and the strain wave gearing are advantageous in protecting against collisions or in regulating force and stiffness.

### BRIEF SUMMARY OF THE DRAWINGS

Further advantages and details of the present disclosure arise from the following description of preferred embodiments with reference to the attached drawing. In the figures:

FIG. 1 shows a side view and a detailed view of a first embodiment of a device according to the present disclosure;

FIG. 2 shows a sectional view and a detailed view of the device shown in FIG. 1;

FIG. 3 shows a plan view of a second embodiment of the device;

FIG. 4 shows a side view of the device shown in FIG. 3;

FIG. 5 shows a sectional view and a detailed view of the side view of the device shown in FIG. 4.

### DETAILED DESCRIPTION

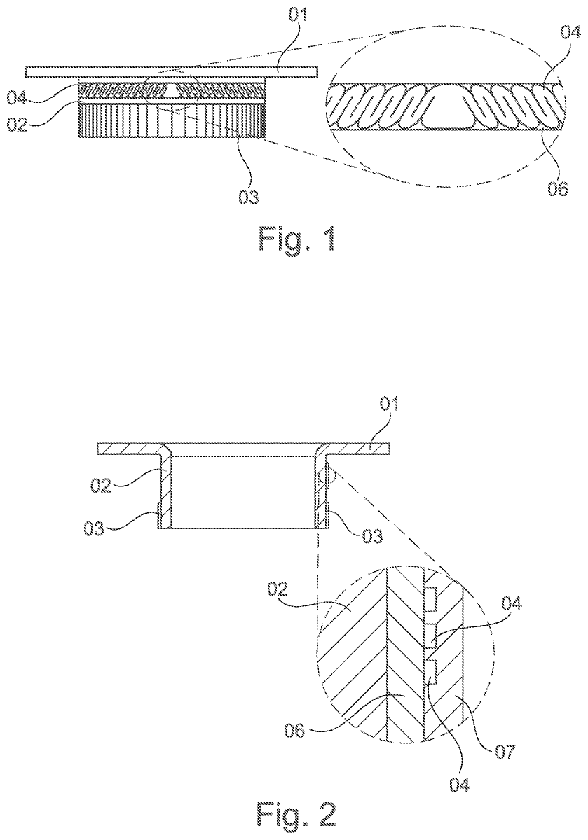

FIG. 1 shows a side view and a detailed view of a first embodiment of a device according to the present disclosure. The device represents a flexible spline usable in a strain wave gearing, wherein the flexible spline consists of a disk 01 and a cylindrical component 02 axially adjoining the disk. Preferably, the flexible spline is made of steel. The cylindrical component 02 or sleeve is arranged on the inner diameter of the disk 01. The cylindrical component 02 has an external toothing 03 on its section facing away from the disk 01. A deformation-sensitive measurement layer 04 in the form of a strain gauge, in particular in the form of a Sensotect strain gauge, is arranged on the section of the outer circumference of the cylindrical component 02 facing the disk 01. An insulating insulation layer 06 is formed between the base material of the cylindrical component 02 and the deformation-sensitive measurement layer 04. A torque of the strain wave gearing is determined by means of the deformation-sensitive measurement layer 04. The measurement layer preferably has a structuring which forms a striped pattern.

Furthermore, a detailed view of the deformation-sensitive measurement layer 04 is shown in FIG. 1. In the example shown, the formed structure of the measurement layer 04 runs in numerous meanders, the axes of the non-curved sections of the structure being inclined to the cylinder axis of the component 02.

One of the advantages of the device according to the present disclosure is that it is designed to save installation space.

FIG. 2 shows a sectional view of the flexible spline shown in FIG. 1 with the disk 01 and the cylindrical component 02. In a detailed view of FIG. 2, the sequence of layers of the device is shown. On the cylindrical component 02, which is made of steel, the insulation layer 06 is applied, on which the deformation-sensitive measurement layer 04 and a protective layer 07 arranged thereon are applied. The deformation-sensitive measurement layer 04 is a structured NiCr functional layer.

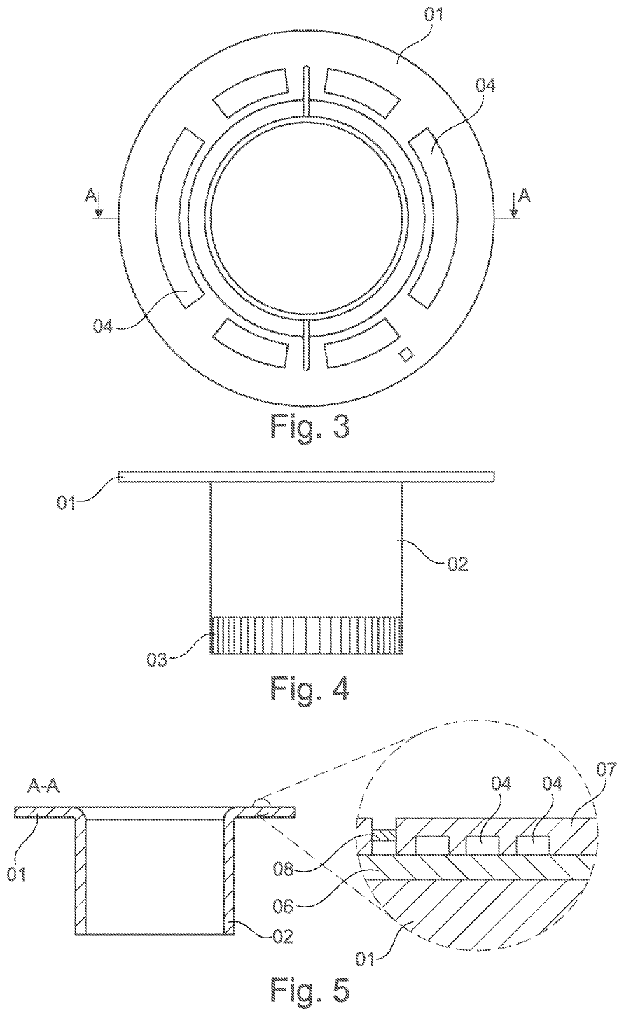

FIG. 3 shows a plan view of a further embodiment of the device. Differing from the device shown in FIG. 1, here the disk 01 has the deformation-sensitive measurement layer 04. No deformation-sensitive measurement layer is formed on the outer circumference of the cylindrical component 02. The individual components of the deformation-sensitive measurement layer 04 are circumferentially distributed on the disk 01. The device is designed here as a collar sleeve.

FIG. 4 shows a side view of the collar sleeve shown in FIG. 3. Since the deformation-sensitive measurement layer 04 is formed on the disk 01, the measurement layer on the outer circumference of the cylindrical component 02 is missing. In the area of the cylindrical component 02 facing away from the disk 01, the toothing 03 is also formed on the outer circumference.

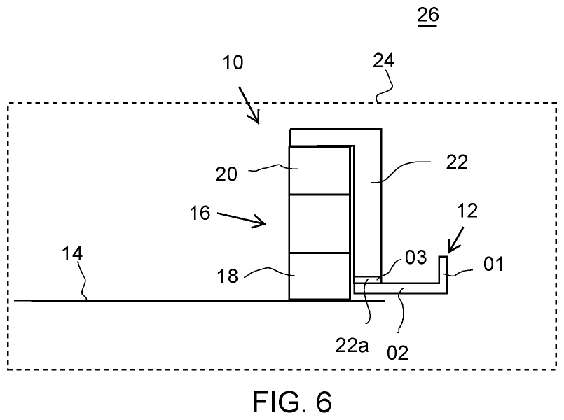

FIG. 5 shows a sectional view of the side view of the device shown in FIG. 4. Furthermore, FIG. 5 shows a detailed view of the sequence of layers of the disk 01. The insulation layer 06, preferably consisting of Al₂O₃, is arranged on the steel disk 01. The deformation-sensitive measurement layer 04 with a protective layer 07 located thereon is arranged on the insulation layer 06. Contact layers 08 for making electrical contact are located between the individual deformation-sensitive measurement layers 04.

FIG. 6 schematically shows a strain wave gearing 10 according to the present disclosure comprises a device 12 for measuring a torque according to the device described with respect to FIG. 1. Further, the strain wave gearing 10 comprises a drive shaft 14, a wave generator 16 which may be a rolling bearing with a non-circular, e.g., elliptical, inner ring 18 and a deformable outer ring 20, a ring gear 22, and an elastic sleeve in the form of the flexible spline 01, 02. The flexible spline 01, 02 exhibits external toothing 03 and the ring gear 22 exhibits internal toothing 22a. The flexible spline 01, 02 and ring gear 22 are arranged coaxially to each other so that the gear teeth 03, 22a mesh with one another. The inner ring 18 of the wave generator 16 is positioned on the drive shaft so that it drives the component. Device 12 may be part of a robot arm or a robot arm joint 24 of a robotics system 26.

### LIST OF REFERENCE SYMBOLS

01 Disk
02 Cylindrical component
03 External toothing
04 Deformation-sensitive measurement layer
06 Insulation layer
07 Protective layer
08 Contact layer
10 Strain wave gearing
12 Device for measuring torque
14 Draft shaft
16 Wave generator
18 Inner ring
20 Outer ring
22 Ring gear
22a Internal toothing
24 Robot arm joint
26 Robotics system

What is claimed is:

1. A device for measuring a torque of a strain wave gearing, the device comprising:
a flexible spline for receiving the torque;
an electrically insulating insulation layer arranged on the flexible spline; and
a deformation-sensitive measurement layer arranged on the electrically insulating insulation layer.

2. The device according to claim 1, wherein the flexible spline is part of a robot arm or a robot arm joint of a robotics system.

3. The device according to claim 1, further comprising a protective layer on the deformation-sensitive measurement layer.

4. The device according to claim 3, wherein the protective layer is organic.

5. The device according to claim 3, wherein a total thickness of a sequence of layers applied to the flexible spline, consisting of the deformation-sensitive measurement layer, the electrically insulating insulation layer and the protective layer, is less than 200 μm.

6. The device according to claim 1, wherein the electrically insulating insulation layer consists of one or more oxide layers and/or a carbon coating.

7. The device according to claim 1, wherein a total thickness of a sequence of layers applied to the flexible spline, consisting of the deformation-sensitive measurement layer and the electrically insulating insulation layer, is less than 20 μm.

8. The device according to claim 1, further comprising further components are arranged on the flexible spline.

9. A strain wave gearing for a robot arm, comprising:
the device for measuring a torque according to claim 1;
a drive shaft;
a wave generator having an inner ring and an outer ring; and
an internally toothed ring gear,
the flexible spline being an externally toothed flexible spline, wherein the externally toothed flexible spline and the internally toothed ring gear are arranged coaxial with respect to each other such that toothings of the externally toothed flexible spline mesh with toothings of the internally toothed ring gear, and wherein the inner ring is positioned on the drive shaft such that the drive shaft drives and deforms the externally toothed flexible spline.

10. The device according to claim 3, wherein the protective layer is inorganic.

11. A method of producing a device for measuring a torque of a strain wave gearing, the method comprising:
providing a flexible spline reconfigured for receiving a torque input;
depositing an electrically insulating insulation layer directly on the flexible spline; and
applying a deformation-sensitive measurement layer directly on the electrically insulating insulation layer.

12. The method as recited in claim 11 wherein the depositing is performed by physical vapor deposition or chemical assisted physical vapor deposition.

13. The method as recited in claim 11 wherein the electrically insulating insulation layer is aluminum oxide and/or wollastonite.

14. The method as recited in claim 11 wherein the deformation-sensitive measurement layer consists of metal or an alloy.

15. The method as recited in claim 11 further comprising structuring the deformation-sensitive measurement layer into a striped pattern.

16. The method as recited in claim 15 wherein the striped pattern includes stripes in an angular range between 35° and 55° with respect to a longitudinal axis of the flexible spine.

17. The method as recited in claim 15 wherein the structuring of the deformation-sensitive measurement layer into the striped pattern is performed by laser structuring or by etching after the applying of the deformation-sensitive measurement layer directly on the electrically insulating insulation layer.

18. A device for measuring a torque of a strain wave gearing, the device comprising:
a flexible spline, the flexible spline including a disk and a cylindrical component adjoining the disk, the cylindrical component including a torque input section configured for receiving the torque;
an electrically insulating insulation layer arranged on the cylindrical component offset from the torque input section; and
a deformation-sensitive measurement layer arranged on the electrically insulating insulation layer.

19. The device as recited in claim 18 wherein the deformation-sensitive measurement layer consists of metal or an alloy structured into a striped pattern including numerous meanders and non-curved sections forming stripes, axes of the stripes of the structure being inclined with respect to a longitudinal axis of the flexible spine.

20. The device as recited in claim 19 wherein the stripes are inclined in an angular range between 35° and 55° with respect to the longitudinal axis of the flexible spine.
